(19) United States
(12) Patent Application Publication
(10) Pub. No.: US 2024/0392006 A1
(43) Pub. Date: Nov. 28, 2024

(54) FORMULATIONS OF IMMUNE CHECK POINT INHIBITORS

(71) Applicant: DR. REDDY’S LABORATORIES LIMITED, Hyderabad (IN)

(72) Inventors: Murali JAYARAMAN, Kancheepuram (Distt) (IN); Saisharan K GOUD, Kamareddy (Distt) (IN); Sunil ASHOK NANKAR, Jalgaon (Distt) (IN); Asif RASUL SAYYAD, Ahmednagar (IN); Suman LABALA, Srikakulam (IN); Mahesh INGALE, Solapur (IN)

(21) Appl. No.: 18/689,296

(22) PCT Filed: Sep. 7, 2022
(86) PCT No.: PCT/IN2022/050801

§ 371 Date: Mar. 5, 2024

(30) Foreign Application Priority Data
Sep. 7, 2021 (IN) 202141040560

(51) Int. Cl. C07K 16/28; A61K 47/02; A61K 47/18; A61K 47/26

(52) U.S. Cl. CPC C07K 16/2818; A61K 47/02; A61K 47/183; A61K 47/26; C07K 2317/94

(57) ABSTRACT

The present invention relates to pharmaceutical formulations of antibodies and antigen-binding fragments against human programmed death receptor-1 (PD-1) or programmed death receptor Ligand 1 (PD-L1), and method for preparing the same. The disclosed formulations are free of sugar or sugar alcohol and stabilizes anti-PD1/anti-PD L1 antibody from lower to higher concentrations rendering it suitable for different modes of administration (subcutaneous/intravenous).

### FIELD OF THE INVENTION

The present invention relates to the field of therapeutic antibody formulations. More specifically, the present invention relates to the field of stable formulations of antibodies and antigen-binding fragments against human programmed death receptor-1 (PD-1) (or) programmed death receptor Ligand 1 (PD-L1), and method for preparing the same.

### BACKGROUND

Over the past two decades, recombinant DNA technology has led to the commercialization of many proteins, particularly antibody therapeutics. The effectiveness of these therapeutic antibodies is majorly dependent on the stability, route of administration and their dosage forms and concentrations. This in turn, necessitates therapeutic antibodies to be formulated appropriately to retain the stability and activity of a therapeutic antibody.

Formulations for each route of administration and dosage forms may be unique and, therefore, have specific requirements. Solid dosage forms, such as lyophilized powders, are generally more stable than liquid (aqueous) formulations. However, reconstitution of the lyophilized formulation requires a significant vial overfill, care in handling and involves high production cost relative to a liquid formulation. While liquid formulations are advantageous in these and are usually preferred for injectable protein therapeutics (in terms of convenience for the end user and ease of preparation for the manufacturer), this form may not always be feasible given the susceptibility of proteins to denaturation, aggregation and oxidation under stresses such as temperature, pH changes, agitation etc., All of these stress factors could result in the loss of biological activity of a therapeutic protein/antibody.

Hence, it is necessary to formulate an antibody in an appropriate buffer and also selection of excipients also impact stability of the antibody in a formulation. The stability of an antibody in liquid formulation depends not only on the kinds of excipients used in the formulation, but also on the amounts and proportions of the excipients relative to one another. Apart from these, other factors such as viscosity of the formulation, visual appearance must also be taken care while preparing any formulation.

Antibodies which binds to the human programmed death-1 protein (PD-1) or human programmed death ligand-1 protein (PDL-1) are one of the examples of therpaeutic antibodies and gained lot of importance due to it's broad spectrum in treating various oncological disorders.

Although anti-PD-1 or anti-PD-L1 antibodies are known, there remains a need in the art for novel pharmaceutical formulations comprising anti-PD-1 or anti-PDL1 antibodies that are sufficiently stable and suitable for administration to patients.

### SUMMARY

The present invention discloses a pharmaceutical formulation of an anti-PD-1 or an anti-PD-L1 antibody formulation

The invention presents a formulation of an anti-PD-1 or an anti-PD-L1 antibody or an antigen-binding fragment thereof, comprising an anti-PD-1 or an anti-PD-L1 antibody, a buffer having pH of about 4.5 to about 6.5 and pharmaceutically acceptable polymer.

In another aspect, the invention presents a pharmaceutical formulation of an anti-PD-1 or an anti-PD-L1 antibody or an antigen-binding fragment thereof, comprising an anti-PD-1 or an anti-PD-L1 antibody, a buffer having pH of about 4.5 to about 6.5, and comprising polyethylene glycol (PEG) or propylene glycol and wherein the formulation is free of a sugar or sugar alcohol. The formulation further includes one or more pharmaceutically acceptable stabilizers.

The disclosed formulations of the present invention stabilizes anti-PD1 or anti-PD-L1 antibody from lower to higher concentration (i.e; from 10 mg/ml to 200 mg/ml) and suitable for intravenous as well as subcutaneous formulations.

In one aspect, the invention discloses a method of controlling particle formation and/or formation of charge variants and/or aggregation and/or fragmentation and/or deamidation of an anti-PD1 or an anti-PD-L1 antibody in an anti-PD-1 or an anti-PD-L1 antibody composition wherein the method comprises addition of, succinate buffer or citrate buffer or acetate buffer or histidine buffer, or it's derivatives or salts or combination thereof, and PEG or propylene glycol, to the antibody composition. The said buffer composition can be added during pre-formulation and/or at formulation stage of the antibody production.

In addition, the invention discloses a method of controlling opalescence of an anti-PD1 or an anti-PDL1 antibody composition in its composition, wherein the method comprises addition of, succinate buffer or citrate buffer or acetate buffer or histidine buffer or it's derivatives or salts or combination thereof, and PEG or propylene glycol to the antibody composition. The said buffer composition can be added during pre-formulation and/or at formulation stage of the antibody production to maintain the antibody in soluble form in the composition, thereby maintaining opalescence. Further, the opalescence of the formulations obtained from the said process matches with reference opalescence standard (ROS) II or II-III.

The invention also discloses a method to impart colloidal stability to an anti-PD1 or an anti-PDL1 antibody wherein the method comprises formulating the anti-PD1 or anti-PD-L1 antibody in a buffer composition comprising succinate buffer or citrate buffer or acetate buffer or histidine buffer or it's derivative or salts or combination thereof, and PEG or propylene glycol. The pH of the formulation of the present invention can range from about 4.5 to about 6.5.

The disclosed formulations of the invention exhibit stability under at least one of the following accelerated conditions that includes a temperature ranging from 25° C. to 40° C. and for a period of time ranging from 1 day to 28 days/4 weeks. The antibody in the said formulation is stable and maintains 98% or more (≥98%) of monomeric content of the antibody in the formulation even after storage for two weeks at 40° C.

### DETAILED DESCRIPTION OF THE INVENTION

### Definitions

The term “about” used herein would mean and include a variation of upto 20% from the particular value.

The term “antibody” as used herein encompasses whole antibodies or an antigen binding fragment (i.e., “antigen-binding portion”) or fusion protein thereof.

The term “stable” formulation refers to the formulation wherein the antibody therein retains its physical stability and/or chemical stability and/or biological activity upon storage.

The term “buffer” used herein refers to an agent which resists to any change in pH of a solution, near a chosen value, up on addition of acid or base. The buffer herein includes buffering agents, or its' derivative, or salts and combination thereof.

Stability studies provides evidence of the quality of an antibody under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the antibody in the pharmaceutical formulations. An antibody “retains its physical stability” in a pharmaceutical formulation if it shows substantially no signs of aggregation, precipitation and/or denaturation upon visual examination of color and/or clarity, or as measured by UV light scattering or by size exclusion chromatography. An antibody is said to “retain its chemical stability” in a pharmaceutical formulation when its shows no or minimal formation of product variants which may include variants as a result of chemical modification of antibody of interest such as deamination, oxidation etc. Analytical methods such as ion exchange chromatography and hydrophobic ion chromatography may be used to investigate the chemical product variants.

The term ‘monomer’ as used herein describes antibodies consisting of two light chains and two heavy chains. The monomer content of an antibody composition is typically analyzed by size exclusion chromatography (SEC). As per the separation principle of SEC the large molecules or molecules with high molecular weight (HMW) elute first followed by smaller or lower weight molecules. In a typical SEC profile for an antibody composition, aggregates that may include dimers, multimers, etc., elute first, followed by monomer, and the clipped antibody variants or degradants may be eluted last. In some circumstances the aggregate peak or the degradant peaks may not elute as a baseline separated peaks but instead as a shoulder or abnormal broad peaks. In order to maintain the appropriate activity of an antibody, in particular of a therapeutic antibody, it is desirable to reduce the formation of aggregate or fragmentation of products and hence control the monomer content to a target value. Ability to inhibit the formation of aggregate and degradant content as measured at various time points during stability studies may indicate the suitability of the candidate formulation for antibody of interest. TSK-GEL G3000SWXL (7.8 mm×30 cm) column from TOSCH can be used on water HPLC to perform SEC.

The term ‘main peak’ as used herein refers to the peak that elutes in abundance (major peak) during a cation exchange chromatography. The peak that elutes earlier than the main peak, during a cation exchange chromatography, with a charge that is acidic relative to the main peak is termed acidic variant peak. The peak that elutes later than the main peak, during a cation exchange chromatography, with a charge that is relatively basic than the main peak is termed as basic variant peak. The main peak content can be determined by Ion exchange chromatography (IEC). There are two modes of IEC available viz., cation and anion exchange chromatography. Negatively charged molecules bind to anion exchange resins while positively charged molecules bind to cation exchange resins. In a typical cation exchange chromatographic profile of an antibody composition acidic variants elute first followed by the main peak and thereafter lastly the basic variants will be eluted. The acidic variants are a result of antibody modifications such as deamidation of asparagine residues. The basic variants are a result of incomplete removal of C-terminal lysine residue(s). In general, in an antibody a lysine residue is present at the C-terminal end of both heavy and light chain. An antibody molecule containing lysine at both heavy and light chain is referred to as K2 variant, the antibody molecule containing lysine residue at either one of heavy and light chain is referred to as K1 variant and antibody molecule having none is K0 molecule. Carboxypeptidase B (CP-B enzyme) enzyme acts on the C-terminal lysine residues present on K2 and K1 variants and thus converting them as K0 molecules. As per circumstances of the case, the IEC analysis can be carried out for samples digested with carboxypeptidase B (CP-B) enzyme. In a typical stability study it is expected that a stable formulation leads to reduction in formation of charge variants (acidic and basic variants), during the study, and hence minimize any reduction in main peak content.

Pharmaceutically acceptable excipients refer to the additives or carriers, which may contribute to stability of the antibody in formulation. The excipients may encompass stabilizers and tonicity modifiers. Examples of stabilizers and tonicity modifiers include, but not limited to, salts, surfactants, and derivatives and combination thereof. The at least one stabilizer in a pharmaceutical formulation of the present invention can be a polyethylene glycol (PEG), β-cyclodextrin or equivalents thereof.

The term “sugar/s” as used herein includes organic compounds having general formula of all carbohydrates of the general formula Cn(H2O)n. Sugars can be referred to monosaccharides, disaccharides, and polysaccharides. Examples of sugars include, but are not limited to, sucrose, trehalose, glucose, dextrose, raffinose and others.

The term “sugar alcohol” as used herein are derivatives of sugar containing one hydroxyl group attached to each carbon atom. Examples of sugar alcohols include mannitol, sorbitol, xylitol etc., The term sugar alcohol herein is also used interchangeably with ‘polyol’.

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

Examples of salts include, but not limited to, sodium chloride, potassium chloride, magnesium chloride, sodium thiocyanate, ammonium thiocyanate, ammonium sulfate, ammonium chloride, calcium chloride, zinc chloride and/or sodium acetate.

The term “opalescence” or “opalescent appearance” refers to the degree of turbidity detected in a solution, e.g., a protein preparation, as a function of the concentration of one or more of the components in the solution, e.g., protein and/or salt concentration. The degree of turbidity can be calculated by reference to a standard curve generated using suspensions of known turbidity. Reference standards for determining the degree of turbidity for pharmaceutical compositions can be based on the United States Pharmacopeia or European Pharmacopeia criteria. Here, in this invention to measure opalescence, first Formazine solution has been prepared by mixing equal volumes of a hydrazine sulfate solution and hexamethylenetetramine solution and then diluted to prepare various reference opalescence standards. The opalescence standards includes ROS-I, ROS-II, ROS-III and ROS-IV.

Nephelometry is a turbidometric method used to detect the presence of soluble aggregates or to indicate opalescence. The output is listed in terms of nephelometric turbidity units (NTUs).

“Pre-formulation steps” refers to any or multiple steps performed before formulating the protein into a therapeutic product. Examples of such steps include, chromatography, filtration, (ultrafiltration, sterile filtration, nano filtration, diafiltration, tangential flow filtration, depth filtration), or any other steps performed to concentrate the protein or to exchange the buffer to a different/suitable buffer. The filtration steps mentioned herein may be performed in a tangential flow filtration mode.

“Formulation steps” refers to steps which are followed after the downstream chromatographic and filtration steps to prepare drug product from drug substance, the latter obtained from the pre-formulation steps.

The term “chelators/chelating agents” refers to a compound which can form at least one bond with a metal atom. A chelating agent is typically a multidentate ligand that can be used in compositions as a stabilizer to complex with species, which might otherwise promote instability. Exemplary chelating agents include aminopolycarboxylic acids, hydroxyaminocarboxylic acids, N-substituted glycines, 2-(2-amino-2-oxocthyl)aminoethane sulfonic acid (BES), deferoxamine (DEF), niacinamide, desoxycholates, ethylenediaminetetraacetic acid (EDTA), diethylenetriaminepentaacetic acid (DTPA), nitrilotriacetic acid (NTA), N-2-acetamido-2-iminodiacetic acid (ADA), bis(am inoethyl)glycolether, N,N,N′,N′-tetraacetic acid (EGTA), trans-diaminocyclohexane tetraacetic acid (DCTA), N-hydroxyethyliminodiacetic acid (HIMDA), N,N-bis-hydroxyethylglycine (bicine), N-(trishydroxymethylmethyl)glycine (tricine), glycylglycine, sodium desoxycholate, ethylenediamine; propylenediamine; diethylenetriamine; triethylenetetraamine (trien), ethylenediaminetetraaceto EDTA; disodium EDTA, EDTA, calcium EDTA oxalic acid and malate.

The term “antioxidant” refers to an agent that inhibits the oxidation of other molecules and is not part of buffer component. Examples of antioxidants herein include citrate, methionine, lipoic acid, uric acid, glutathione, tocopherol, carotene, lycopene, cysteine, phosphonate compounds, e.g., etidronic acid, desferoxamine and malate.

### Detailed Description of the Embodiments

The present invention discloses a stable pharmaceutical formulation of an anti-PD1 or an anti-PDL1 antibody stabilized using poly ethylene glycol (PEG) or propylene glycol. PEG or propylene glycol based formulation of the present invention addresses a specific problem in the art. Wherein, most of the approved anti-PD1 or anti-PDL1 antibody formulations comprises either sugar or sugar alcohol. However, these sugar/polyols prone for hydrolysis, may lead to maillard reaction and may impact the long term stability of the antibody. Some of the polyols (i.e., mannitol) are specifically known for freeze-thaw induced instability due to crystallization. Further, increase in the concentration of sugar/polyol leads to increase in formulation viscosity (>20 cP), which is a concern of injection site pain. The invention disclosed achieves stability of the antibody without the need for inclusion of sugar or sugar alcohols.

In one embodiment, the invention discloses a pharmaceutical formulation of an antibody against human programmed cell death protein 1 (PD-1) or programmed death receptor Ligand 1(PDL1) comprising:
(i) an anti-PD-1 or an anti-PDL1 antibody,
(ii) a buffer having pH of about 4.5 to about 6.5
(iii) one or more stabilizers;
(iv) a surfactant and,
wherein the formulation is free of any sugar or sugar alcohol.

In the above embodiment, the one or more stabilizer is PEG or propylene glycol.

In any of the above said embodiments, the buffer mentioned in the formulation is an organic buffer or an inorganic buffer, and/or combination thereof.

In the above mentioned embodiment of the invention, the said organic buffer is a succinate buffer or an acetate buffer or a citrate buffer or a histidine buffer and the inorganic buffer mentioned in the formulation is phosphate buffer.

In an embodiment, the invention discloses a method of imparting colloidal stability to an anti-PD1 or an anti-PD L1 antibody, in an anti-PD1 or an anti-PDL1 antibody composition, wherein the method involves addition of a buffer composition comprising succinate buffer or citrate buffer or acetate buffer or histidine buffer or it's derivatives or salts or combination thereof and PEG or propylene glycol to the antibody composition during pre-formulation and/or formulation stage of the antibody production, wherein the said buffer composition does not contain any sugar or sugar alcohol.

In an embodiment, the invention discloses a pharmaceutical formulation of an antibody against human programmed cell death protein 1 (PD-1) or programmed death receptor Ligand 1(PDL1) comprising:
(i) an anti-PDL1 or an anti-PDL1 antibody,
(ii) succinate buffer or citrate buffer or acetate buffer or histidine buffer
(iii) one or more stabilizers;
(iv) a surfactant and;
   wherein the one or more stabilizer is not a sugar or sugar alcohol and the formulation is free of any sugar or sugar alcohol.

In another embodiment, the invention discloses a method of controlling particle formation in an anti-PD-1 or an anti-PD-L1 antibody composition wherein the method comprises addition of a buffer composition comprising succinate buffer or citrate buffer or acetate buffer or histidine buffer and PEG or propylene glycol, to the antibody composition during pre-formulation and/or formulation stage of the antibody production.

In yet another embodiment, the invention discloses a method of controlling formation of charge variants in an anti-PD1 or an anti-PD-L1 antibody composition wherein the method comprises addition of a buffer composition comprising succinate buffer or citrate buffer or acetate buffer or histidine buffer and PEG or propylene glycol, to the antibody composition during pre-formulation and/or formulation stage of the antibody production.

In an embodiment, the invention discloses a method of controlling aggregation and/or fragmentation of an anti-PD1 or an anti-PD-L1 antibody composition wherein the method comprises addition of a buffer composition comprising succinate buffer or citrate buffer or acetate buffer or histidine and PEG or propylene glycol, to the antibody composition during pre-formulation and/or formulation stage of the antibody production.

In some of the embodiments, the buffer composition comprising succinate buffer or it's derivatives or salts or combination thereof, is a succinate buffer or an arginine-succinate buffer or a succinate-phosphate buffer. The succinate buffer composition may also further contain at least one or more pharmaceutically acceptable excipients/stabilizer but, the buffer composition does not include any sugar or sugar alcohol. The said composition can be added during pre-formulation and/or formulation stage of the antibody production.

In some of the embodiments, the buffer composition comprising citrate buffer or it's derivative or salts is a citrate buffer, or a citrate-arginine buffer, or a histidine-citrate buffer, or a citrate-phosphate buffer. The citrate buffer composition may also further contain at least one pharmaceutically acceptable excipients/stabilizer but does not include any sugar or sugar alcohol. The said composition can be added during pre-formulation and/or formulation stage of the antibody production.

In some of the embodiments, the buffer composition comprising an acetate buffer or it's derivatives or salts composition is an acetate buffer or an arginine-acetate buffer, or an acetate-phosphate buffer. The acetate buffer composition may also further contain at least one pharmaceutically acceptable excipients/stabilizer but does not include any sugar or sugar alcohol. The said composition can be added during pre-formulation and formulation stage of the antibody production.

In some of the embodiments, the buffer composition comprising histidine buffer or it's derivative or salts or combination thereof is a histidine buffer, or a histidine-citrate buffer, or a histidine-phosphate buffer. The histidine buffer composition may also further contain at least one pharmaceutically acceptable excipients/stabilizer but does not include any sugar or sugar alcohol. The said composition can be added during pre-formulation and/or formulation stage of the antibody production.

In any of the above mentioned embodiments, the one or more pharmaceutically acceptable excipients/stabilizers is β-cyclodextrin or salt or amino acid. The one or more stabilizers in any of the above mentioned embodiments do not include any sugar or sugar alcohol.

In an embodiment, the invention discloses a formulation of an anti-PD-1 or an anti-PD-L1 antibody or an antigen-binding fragment thereof, comprising an anti-PD-1 or an anti-PD-L1 antibody, a buffer having pH of about 4.5 to about 6.5 and pharmaceutically acceptable polymer.

In an embodiment, the invention discloses a pharmaceutical formulation of an anti-PD-1 or an anti-PDL1 antibody comprising:
(i) an anti-PD-1 or ananti-PDL1 antibody
(ii) a buffer having pH of about 4.5 to about 6.5;
(iii) poly ethylene glycol (PEG) or propylene glycol or β-cyclodextrin or equivalents thereof,
(iv) a surfactant; and
   wherein the formulation is free of sugar or sugar alcohol.

In an embodiment, the invention discloses a pharmaceutical formulation of an anti-PD-1 or an anti-PDL1 antibody comprising:
(i) an anti-PD-1 or an anti-PDL1 antibody
(ii) a buffer having pH of about 4.5 to about 6.5;
(iii) poly ethylene glycol (PEG) or propylene glycol or β-cyclodextrin or equivalents thereof,
(iv) salt
(iv) a surfactant; and
   wherein the formulation is free of sugar or sugar alcohol.

In an embodiment, the invention discloses a pharmaceutical formulation of an anti-PD-1 or an anti-PDL1 antibody comprising:
(i) an anti-PD-1 or an anti-PDL1 antibody
(ii) a buffer having pH of about 4.5 to about 6.5;
(iii) poly ethylene glycol (PEG) or propylene glycol or β-cyclodextrin or equivalents thereof,
(iv) amino acid
(iv) a surfactant; and
   wherein the formulation is free of sugar or sugar alcohol.

In any of the above mentioned embodiment, the amino acid is arginine or glycine.

In any of the above mentioned embodiments, the salt is sodium chloride.

In any of the above mentioned embodiments, the surfactant is polysorbate-80 or polyosrbate-20.

The disclosed formulations of the present invention stabilize an anti-PD1 or anti-PD-L1 antibody at various concentration levels i.e., from about 10 mg/ml to about 200 mg/ml and are suitable for intravenous as well as subcutaneous routes of administration. Further, viscosity of the anti-PD1 or anti-PD-L1 antibody formulations is less than 20 cP, specifically less than 10 cP.

In an embodiment, the invention discloses a pharmaceutical formulation of an anti-PD1 antibody or an anti-PD L1 antibody comprising:
i) an anti-PD1 or an anti-PD L1 antibody,
ii) 10-50 mM succinate—buffer
iii) poly ethylene glycol (PEG) or propylene glycol or β-cyclodextrin or equivalent thereof,
iv) a surfactant, and
wherein the formulation does not contain any sugar or sugar alcohol.

In any of the above mentioned embodiments, the claimed anti-PD1 or-anti-PD-L1 antibody formulations of the invention exhibit stability under at least one of the following conditions, wherein the temperature range from 25° C. to 50° C. for a period of time which includes from 1 day to 6 months.

In any of the above mentioned embodiments, the anti-PD1 antibody is nivolumab, pembrolizumab, cemiplimab or dosrtalimab.

In any of the above mentioned embodiment, the anti-PDL1 antibody is atezolizumab, avelumab or durvalumab.

In one aspect, the invention discloses a pharmaceutical formulation of nivolumab, comprising nivolumab, succinate buffer having pH of 5.0 to 6.0, 0.5% PEG and surfactant, wherein the formulation is free of sugar or sugar alcohol.

In one aspect, the invention discloses a pharmaceutical formulation of pembrolizumab, comprising pembrolizumab, acetate buffer having pH of 5.0 to 6.0, propylene glycol and surfactant, wherein the formulation is free of sugar or sugar alcohol.

In the above mentioned embodiment, the formulation further comprises an amino acid, arginine. In all of the above mentioned embodiments of the invention, the concentration of the antibody in the formulation is about 10 mg/ml to about 200 mg/ml. Preferably, the concentration of the antibody in the formulation is 10 mg/ml, or 25 mg/ml, or 50 mg/ml, or 60 mg/ml, or 70 mg/ml, or 80 mg/ml, 90 mg/ml, or 100 mg/ml, or 110 mg/ml, or 120 mg/ml, or 150 mg/ml or 160 mg/ml, or 170 mg/ml or 175 mg/ml or 180 mg/ml or 190 mg/ml or 195 mg/ml or 200 mg/ml.

In any of the above mentioned embodiments, the pH of the disclosed formulation of the present invention is in the range from about 4.5 to about 6.5.

In any of the above mentioned embodiments, the pH of the disclosed formulation of the present invention is in the range from about 5.0 to about 6.0.

In any of the above mentioned embodiments, the pH of the disclosed formulation of the present invention is 6.0±0.2.

In any of the above said embodiments of the invention, the anti-PD1 or anti-PD-L1 antibody formulation is stable and contains less than 0.9% of high molecular weight (HMW) species or fragments in the formulation, even after storage at 40° C. for one week.

In any of the above mentioned embodiments, the anti-PD1 or anti-PDL1 antibody formulation's osmolality is less than 600 mOsm/kg, preferably less than 300 mOsm/kg.

The anti-PD1 or anti-PDL1 antibody formulations disclosed in the invention are biologically active.

In any of the above mentioned embodiments, the formulation of anti-PD1 or anti-PD L1 antibody is a stable liquid (aqueous) formulation, which can be used for parenteral administration. Parenteral administration includes intravenous, subcutaneous, intra peritoneal, intramuscular administration or any other route of delivery generally considered to be falling under the scope of parenteral administration and as is well known to a skilled person.

In any of the above embodiments of the invention, the stable liquid/aqueous formulation is suitable and can be lyophilized as lyophilized powders. Further, the lyophilized formulation of anti-PD1 or anti-PDL1 antibody can be reconstituted with appropriate diluent to achieve the liquid formulation suitable for administration.

In any of the above mentioned embodiments, the liquid/aqueous anti-PD1 or anti-PD-L1 antibody formulations are compatible with lyophilization process and the lyophilization process does not impact quality attributes of the antibody.

Another aspect of the invention provides a vial, pre-filled syringe or autoinjector device, or any other suitable device comprising any of the subject formulations described herein. In certain embodiments, the aqueous formulation stored in the vial or pre-filled syringe or an auto injector device comprises anti-PD1/anti-PDL1 antibody, a succinate buffer or an acetate buffer or a citrate buffer or a histidine buffer or it's derivatives or combination thereof, one or more stabilizers comprise poly ethylene glycol (PEG), or propylene glycol or β-cyclodextrin or salt, and surfactant.

Certain specific aspects and embodiments of the invention are more fully described by reference to the following examples. However, these examples should not be construed as limiting the scope of the invention in any manner.

### Examples

### Example 1: Anti-PD1 Antibody Formulations

As part of experimental design, an anti-PD1 antibody, nivolumab antibody expressed in Chinese hamster ovarian (CHO) cell and then purified using chromatographic steps. Purified nivolumab antibody approximately 25 mg/ml in citrate buffer back ground obtained from downstream chromatographic step. Post which, all the samples were buffer exchanged with succinate buffer. And, combination of various excipients such as salt, PEG, and surfactant were added to nivolumab in succinate buffer and the concentration was adjusted to 10 mg/ml. Further, some of the nivolumab antibody samples are formulated in succinate buffer with sugars. Compositions of all nivolumab formulations are given in Table 1.

Post which, all the samples were measured for their particle formation, opalescence, high molecular weight [HMW] species using size exclusion chromatography. To measure opalescence, various USP reference opalescence standards were prepared by diluting primary opalescence solution comprising formazin suspension having 4000 NTU ((Nephelometric Turbidity Units).

All nivolumab formulations were subjected for accelerated stability studies at 40° C. for four weeks and at 25° C. for four weeks. Post which, the samples were analyzed for low molecular weight (LMW) species, high molecular weight species (HMW) and monomer content using size exclusion chromatography (SEC) [results are given in Table 2] and also checked for particle formation, and opalescence [Table 3].

TABLE 1
Compositions of various anti-PD1 antibody
formulations prepared as per Example-1

| Sample | |
|---|---|
| Name | Composition |
| N1 | 10 mg/ml nivolumab, 20 mM succinate buffer, 3% mannitol, |
| | 2.92 mg/mL NaCl, 0.2 mg/mL polysorbate 80, pH 6.0. |
| N2 | 10 mg/ml nivolumab, 20 mM succinate buffer, 2.92 mg/mL |
| | NaCl, 0.2 mg/mL polysorbate 80 |
| N3 | 10 mg/ml nivolumab, 20 mM succinate buffer, 0.5% PEG |
| | 0.2 mg/mL polysorbate 80 |
| N4 | 10 mg/ml nivolumab, 20 mM succinate buffer, 0.5% PEG |
| | 2.92 mg/mL NaCl, 0.2 mg/mL polysorbate 80 |

TABLE 2
Measurements of particle formation of anti-
PD1 antibody formulations as per example 1

Visible particle count per 1 ml

| Sample name | T0 | T4 W at 40° C. |
|---|---|---|
| N1 | 10 | 25 |
| N2 | 15 | 25 |
| N3 | 15 | 20 |
| N4 | 15 | 20 |

T0—indicates data at zero time point;
W—indicates weeks

TABLE 3
SEC data of anti-PD1 antibody formulations
prepared as per example-1

| | SEC data at 40° C. | | | | | |
|---|---|---|---|---|---|---|
| | % Monomer at | | | | | |
| Sample | 40° C. | | % HMW at 40° C. | | % LMW at 40° C. | |
| name | T0 | T4W | T0 | T4W | T0 | T4W |
| N1 | 99.6 | 98.9 | 0.4 | 1 | ND | 0.1 |
| N2 | 99.5 | 98.6 | 0.5 | 1.1 | ND | 0.3 |
| N3 | 99.5 | 98.7 | 0.5 | 1.1 | ND | 0.2 |
| N4 | 99.5 | 98.7 | 0.5 | 1.1 | ND | 0.3 |

T0—indicates data at zero time point;
W—indicates weeks;
ND—not detected

TABLE 4
Opalescence of anti-PD1 antibody formulations
prepared as per example 1

| | Opalescence at 40° C. | | Opalescence at 25° C. |
|---|---|---|---|
| Sample Name | T0 W | T4 W | T4 W |
| N1 | ROS II | ROS II | ROS II |
| N2 | ROS II | ROS II | ROS II |
| N3 | ROS II | ROS II | ROS II |
| N4 | ROS II | ROS II | ROS II |

T0—indicates data at zero time point;
W—indicates weeks

All the above formulations were also checked for change in pH. It was observed that there is no change in pH of the formulations even after storage for four weeks at 40° C. And all the samples were colorless even after storage at 40° C. for four weeks. Osmolality of all the formulations were found to be less than 350 mOsm/kg.

In another experiment, another anti-PD1 antibody pembrolizumab expressed in CHO cells and then purified. Approximately 25 mg/ml pembrolizumab in acetate buffer was obtained from down stream processing steps. Post which, one or more following excipients such as propylene glycol, amino acid and surfactant were added. Few of pembrolizumab samples were formulated with sugar or sugar alcohol. Composition of all pembrolizumab samples are given in Table S.

Post which, the samples were subjected for accelerated stability conditions at 40° C. for four weeks. And, the samples were measured for change in size variants using size exclusion chromatography and charge variants using ion exchange chromatography were measured. Results of the study are given in Table 6 to 8.

TABLE 5
Compositions of various pembrolizumab
formulations prepared as per Example-1

| Sample | |
|---|---|
| Name | Composition |
| P1 | 25 mg/ml pembrolizumab, 10 mM acetate buffer, arginine, |
| | 10% trehalose, polysorbate-80 |
| P2 | 25 mg/ml pembrolizumab, 10 mM acetate buffer, arginine, |
| | 1% propylene glycol, polysorbate-80 |
| P3 | 25 mg/ml pembrolizumab, 10 mM histidine-acetate buffer, |
| | arginine, 10% trehalose, polysorbate-80 |
| P4 | 25 mg/ml pembrolizumab, 10 mM histidine-acetate buffer, |
| | arginine, 1% propylene glycol, polysorbate-80 |

TABLE 6
SEC data of P1 to P4 formulations prepared as per example-1

| | SEC data at 40° C. | |
|---|---|---|
| Sample | % Monomer at 40° C. | % HMW at 40° C. |

| name | T0 | T4 W | T0 | T4 W |
|---|---|---|---|---|
| P1 | 98.9 | 98.0 | 1.1 | 1.8 |
| P2 | 98.8 | 97.3 | 1.2 | 2.4 |
| P3 | 98.9 | 97.9 | 1.1 | 1.8 |
| P4 | 98.8 | 97.0 | 1.2 | 2.7 |

T0—indicates data at zero time point;
W—indicates weeks;
ND—not detected

TABLE 7
Opalescence of P1 to P4 formulations prepared as per example 1

| | Opalescence at 40° C. | |
|---|---|---|
| Sample Name | T0 W | T4 W |
| P1 | ROS I | ROS II-III, no visible particles |
| P2 | ROS I to II | ROS II-III, no visible particles |
| P3 | ROS I | ROS II-III, no visible particles |
| P4 | ROS I to ROS II | ROS II-III, no visible particles |

T0—indicates data at zero time point;
W—indicates weeks

1. A pharmaceutical formulation of an anti-PDI antibody or an anti-PD L1 antibody comprising:
i. an anti-PDI or an anti-PD L1 antibody,
ii. 10-50 mM succinate or acetate buffer,
iii. poly ethylene glycol (PEG) or propylene glycol,
iv. a surfactant, and,
wherein the formulation does not contain any sugar or sugar alcohol.

2. The formulation according to claim 1, wherein the antibody concentration ranges from 10 mg/ml to 200 mg/ml.

3. The formulation according to in claim 1, wherein the formulation is stable at 40° C. for four weeks or at 25° C. for four weeks.

4. The formulation according to claim 1, further comprises amino acid or salt.

5. The formulation according to claim 4, wherein the amino acid in arginine.

6. The formulation according to claim 4, wherein the salt is sodium chloride.

7. The formulation according to claim 1, wherein the surfactant is polysorbate-80.

8. The formulation according to claim 1, wherein the anti-PDI antibody is nivolumab or pembrolizumab.

9. The formulation according to claim 1, wherein the anti-PDL1 antibody is atezolizumab or durvalumab.

10. The formulation according to claim 1, wherein the sugar is sucrose, trehalose, glucose, dextrose, or raffinose.

11. The formulation according to claim 1, wherein the sugar alcohol is mannitol, sorbitol, or xylitol.
